(19) United States
(12) Patent Application Publication
(10) Pub. No.: US 2004/0136863 A1
(43) Pub. Date: Jul. 15, 2004

(54) Filtering system including panel with photocatalytic agent

(75) Inventors: Stephen F. Yates, Arlington Heights, IL (US); Norman J. Van Den Bussche, Arlington Heights, IL (US); Shihan Chen, Palatine, IL (US); Peter M. Michalakos, Chicago, IL (US)

Correspondence Address: Honeywell International Inc., Patent Services Group, 101 Columbia Road, Morristown, NJ, 07962, US

(73) Assignee: Honeywell International Inc.

(21) Appl. No.: 10/345,022

(22) Filed: Jan. 14, 2003

(51) Int. Cl. A61L 9/00

(52) U.S. Cl. 422/4; 422/121; 422/122; 422/5; 422/24

(57) ABSTRACT

Systems and methods for cleansing a fluid stream including a support structure positioned generally perpendicular to the fluid stream, the support structure creating an interior space and including a plurality of apertures to allow fluid to flow through the apertures. Also included is at least one expanded sheet positioned within the space, the expanded sheet having a plurality of expanded slits, and a photocatalytic agent coupled to the expanded sheet to oxidize airborne contaminants when exposed to a light source. Pleats may be formed in the expanded sheet to further increase the reactive surface area.

### TECHNICAL FIELD

The present invention generally relates to a fluid cleansing system. In addition, the present invention relates to a fluid cleansing system including a panel coated with a photocatalytic agent.

### BACKGROUND

Individuals are increasingly concerned that the air they breathe in their homes and places of work is contaminated with pollutants that are, for example, irritating or cause unpleasant odors. Airborne contaminants that are particulate in nature and of a sufficient size can be removed using standard mechanical filtration methods, such as a high efficiency particulate arresting (HEPA) filter.

However, airborne contaminants that are smaller in size (e.g, molecular in dimension), such as volatile organic compounds (VOCs), odors and other molecular contaminants, are more difficult to remove. Adsorption by carbon or other adsorbents is one viable technique for removal of these smaller airborne contaminants, but this technique is troublesome because adsorbents become loaded with contaminants and ineffective with time.

Photocatalytic oxidation is a longer-term solution for removing these smaller airborne contaminants. Photocatalytic oxidation involves the cleansing of air using a photocatalytic filter. The photocatalytic filter typically includes one or more panels with a filter media coated with a photocatalytic agent. An ultraviolet lamp is used to illuminate the photocatalytic agent on the panel, and a catalytic reaction is created when airborne contaminants in the air contact the illuminated photocatalytic agent, causing the airborne contaminant to degrade. See X. Fu, W. A. Zeitner, M. A. Anderson, “Applications in Photocatalytic Purification of Air,” Semiconductor Nanoclusters, Studies in Surface Science and Catalysis, v. 103, P. V. Kamat and D. Meisel ed., pp. 445-461 (1996), incorporated herein by reference, for additional information regarding photocatalytic oxidation and its use to cleanse fluid streams such as air.

The efficiency of photocatalytic filters is limited by the amount of the photocatalytic agent that is illuminated by the ultraviolet lamp. Any portion of the photocatalytic agent that is not illuminated does not perform a filtering function because only the illuminated photocatalytic agent is reactive. In current designs, the only method employed to increase the efficiency of photocatalytic filters is to add additional panels or filtering stages. However, such additions increase the size, weight, power consumption, pressure drop, and cost associated with photocatalytic filters.

Therefore, it is desirable to develop photocatalytic filters that increase filtering efficiency while minimizing the size and cost associated with filtering.

### SUMMARY

The present invention generally relates to a fluid cleansing system. In addition, the present invention relates to a fluid cleansing system including a panel coated with a photocatalytic agent.

In one aspect, the invention relates to a panel for a fluid cleansing system, the panel including a support structure positioned generally perpendicular to a fluid stream and defining an interior space, the support structure defining a plurality of apertures to allow fluid to flow through the apertures. At least one expanded sheet is positioned within the space, the expanded sheet defining a plurality of expanded slits, and a photocatalytic agent coupled to the expanded sheet to oxidize airborne contaminants when exposed to a light source.

In another aspect, the invention relates to a system for cleansing a fluid stream, the system including at least two panels positioned generally parallel to one another, perpendicular to the fluid stream, and defining a first space therebetween. Each panel includes a support structure including two support sheets positioned generally parallel to one another, perpendicular to a fluid stream, and defining a second space therebetween, each of the two support sheets defining a plurality of apertures to allow fluid to flow through the apertures, at least one expanded sheet positioned within the second space, the expanded sheet defining a plurality of expanded generally parallel slits, and a photocatalytic agent coupled to the expanded sheet to oxidize airborne contaminants when exposed to a light source. The system also includes an ultraviolet light source positioned in the first space between the two panels.

In yet another aspect, the invention relates to a method for cleansing a fluid stream, including steps of: providing a sheet; cutting a plurality of slits in the sheet; stretching the sheet to expand each of the plurality of generally parallel slits; coating the sheet with a photocatalytic agent; providing a support around the sheet to form a first panel; disposing the first panel perpendicular to the fluid stream; and exposing the first panel to ultraviolet light to activate the photocatalytic agent.

The above summary of the present invention is not intended to describe each disclosed embodiment or every implementation of the present invention. Figures in the detailed description that follow more particularly exemplify embodiments of the invention. While certain embodiments will be illustrated and described, the invention is not limited to use in such embodiments.

### BRIEF DESCRIPTION OF THE DRAWINGS

The invention may be more completely understood in consideration of the following detailed description of various embodiments of the invention in connection with the accompanying drawings, in which:

FIG. 1 is a cross-sectional view of a portion of a heating, ventilation, and air conditioning (HVAC) system;

FIG. 2 is a top view of an example filtering system made in accordance with the present invention;

FIG. 3 is a cross-sectional view taken along line 3-3 of the filtering system shown in FIG. 2;

FIG. 4 is a front, partial cutaway view of a portion of an example photocatalytic panel made in accordance with the present invention;

FIG. 5 is a front view of a portion of an example expanded sheet made in accordance with the present invention;

FIG. 6 is a side, cross-sectional view taken along line 6-6 of the photocatalytic panel shown in FIG. 4;

FIG. 7 is a graph illustrating pressure drop as a function of flow rate for example panels made in accordance with the present invention;

FIG. 8 is graph illustrating ultraviolet light intensity as a function of distance for example panels made in accordance with the present invention;

FIG. 9 is a graph illustrating ultraviolet light intensity as a function of distance for several different filtering configurations;

FIG. 10 is front view of another example filtering system made in accordance with the present invention;

FIG. 11 is a side view of the filtering system shown in FIG. 10;

FIG. 12 is side, cross-sectional view taken along line 12-12 of the photocatalytic panel shown in FIG. 10;

FIG. 13 is a front, exploded partial cutaway view of another example filtering system made in accordance with the present invention; and

FIG. 14 is a cross-sectional view taken along line 14-14 of the filtering system shown in FIG. 13.

While the invention is amenable to various modifications and alternative forms, specifics thereof have been shown by way of example and the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

The present invention generally relates to a fluid cleansing system. In addition, the present invention relates to a fluid cleansing system including a panel coated with a photocatalytic agent.

As used herein, the phrase “airborne contaminant” means any airborne pollutant or other agent or compound, such as, for example, volatile organic compounds (VOCs), bacteria, pesticides, carbon monoxide, ammonia, hydrogen sulfide, odors, etc. Using the filtering systems disclosed herein, including one or more panels coated with a photocatalytic agent and one or more light sources to activate the photocatalytic agent, the amount of airborne contaminants in an air stream can be reduced.

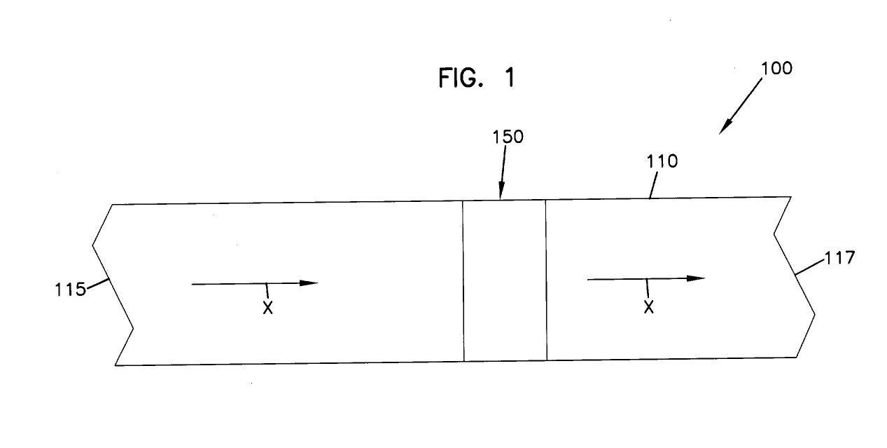

Referring now to FIG. 1, a portion of an example heating, ventilation, and air conditioning (HVAC) system 100 is shown. The system 100 includes a duct 110 through which air flows in a direction X. The system 100 and duct 110 may be part of a ventilation system in aircraft cabins, residences, commercial buildings, and other similar structures where it is desirable to condition the air flowing within the structure.

The system 100 also includes a filtering system 150 disposed within the duct 110. The filtering system 150 is positioned generally perpendicular to the airflow in the direction X within the duct 110 so that the air must pass through the system 150 as the air flows from an intake 115 to an exhaust 117 of the duct. As the air flows through the filtering system 150, airborne contaminants in the air are degraded, as described below.

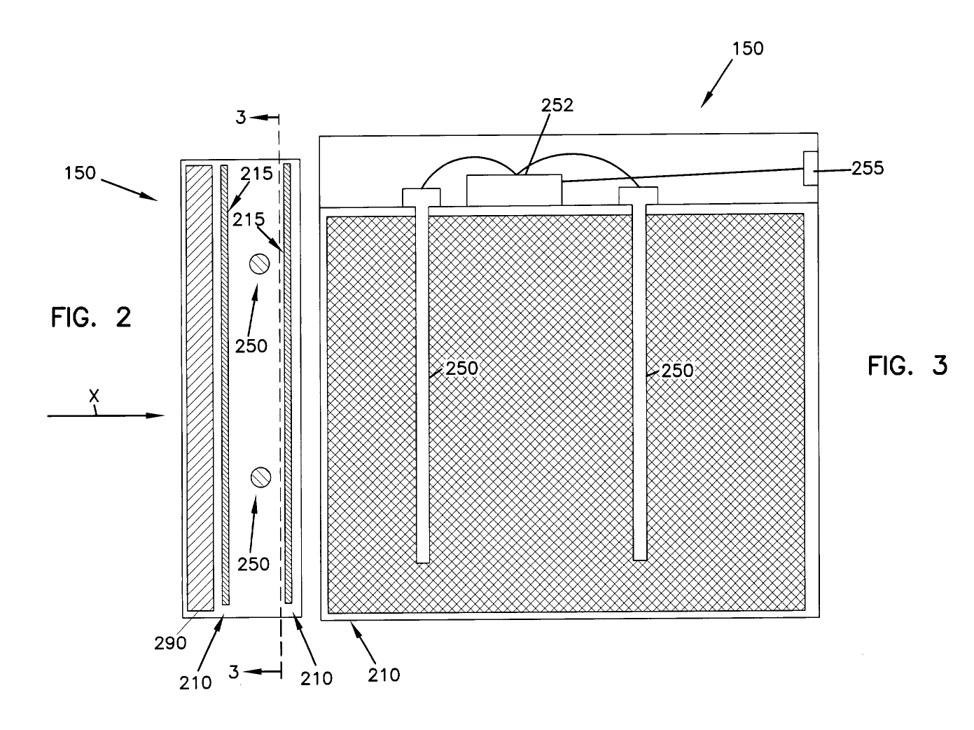

Referring now to FIGS. 2 and 3, the filtering system 150 is shown in greater detail. The system 150 generally includes two ultraviolet lamps 250 positioned between two panels 210. As discussed further herein, alternate embodiments of the system could include varying numbers of lamps and panels. The two panels 210 are each positioned generally perpendicular to the airflow in the direction X so that the air must pass through each panel. Each panel 210 is coated with a photocatalytic agent, as described further below.

The lamps 250 are positioned between the panels 210 to illuminate the surface 215 of each panel with ultraviolet light and thereby activate the photocatalytic agent coated on each panel. In the illustrated embodiment, the lamps 250 are significantly longer than wide and extend along a majority of the height of the panels 210. The lamps 250 may extend along substantially the entire height of the panels to maximize illumination of the surfaces 215. The lamps 250 emit light having a wavelength between 200 and 390 nm. The actual wavelength selected is dependent upon the adsorption range of the photocatalytic agent. In the illustrated embodiment, the lamps 250 preferably have an intensity of at least about 10 μW/cm² at 1 m distance. The quantity of airborne contaminants that are oxidized per unit of time is proportional to the intensity of the lamp, so increased oxidation can be obtained by using a greater intensity lamp. A ballast 252 provides the starting voltage and stabilizes current for the lamps 250, and an on/off switch 255 allows power to the lamps 250 to be turned on and off.

As air passes through each panel 210, airborne contaminants are degraded by oxidation. Oxidation occurs when an airborne contaminant contacts a portion of the photocatalytic agent that has been activated by the UV lamp.

As shown in FIG. 2, also included in the filtering system 150 is a mechanical particulate filter 290 positioned upstream from the panels 210 and lamps 250. The filter 290 functions to remove large airborne contaminants from the air prior to the particulates reaching the panels 210. In other embodiments, the filter 290 can be removed, or additional mechanical filtering stages can be added as desired.

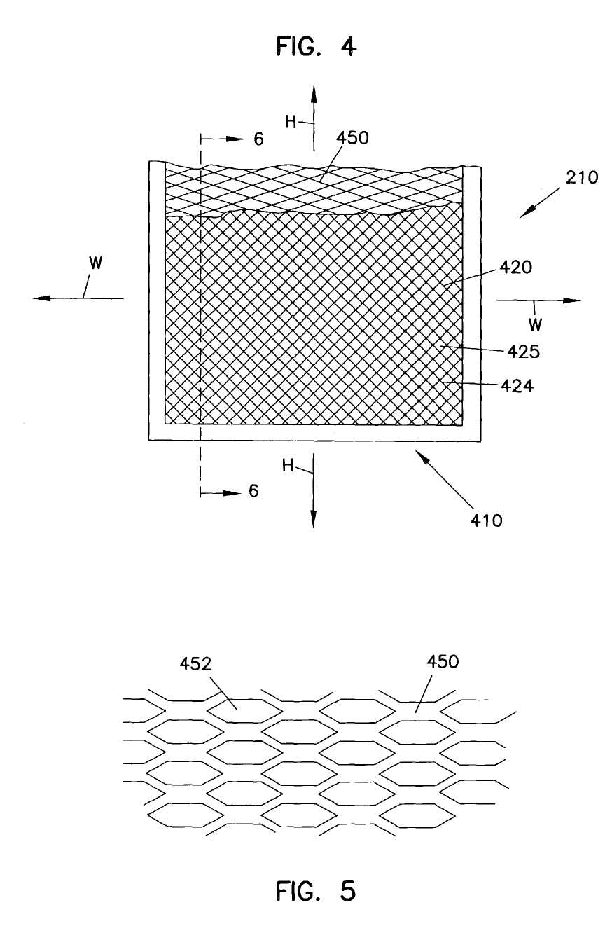

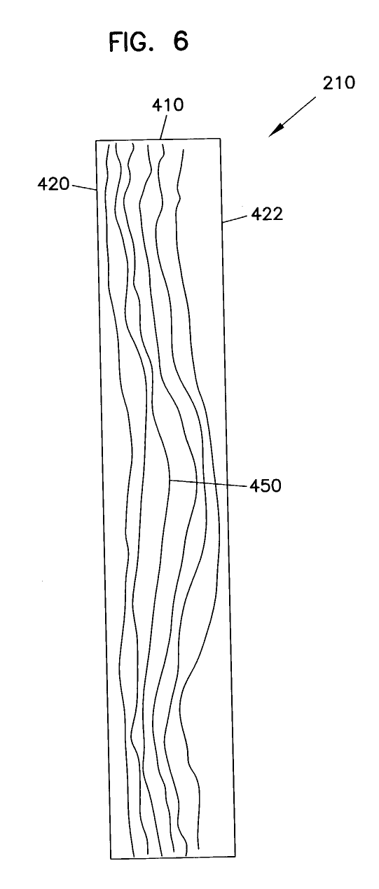

Referring now to FIGS. 4-6, one of the panels 210 is shown in greater detail. The panel 210 in the illustrated embodiment is approximately 20 inches wide by 12 inches tall. Other panel sizes (for example, a 25 inch by 20 inch panel) may be used depending on the size of the duct through which the fluid to be filtered is flowing. The panel 210 is typically sized to substantially occupy the interior dimensions of a duct. The panel 210 includes a support structure comprising a frame 410 and front and rear support surfaces 420 and 422 (FIG. 6). The front and rear support surfaces 420 and 422 include a wire support structure 424 having a plurality of apertures 425 to allow air to flow through the support surfaces 420 and 422.

Disposed within the space defined by the front and rear support surfaces 420 and 422 is a plurality of expanded sheets 450, shown in the top portion of FIG. 4 where the support surface 420 is partially cut away. Each expanded sheet 450 includes a plurality of expanded slits 452, shown in FIG. 5. Each expanded slit 452 is configured to allow air to flow therethrough. The expanded sheets 450 are formed by creating a plurality of slits in each sheet 450, and then stretching the sheet 450 in opposing H and W directions to expand each slit to form the expanded slits 452. In the examples shown, each slit forms a diamond-shaped opening.

In the illustrated embodiment, four expanded sheets are provided, each sheet being made of aluminum. In other embodiments, six expanded sheets are used. More or fewer sheets may be added (e.g., less than ten sheets), as desired, to maximize filtration while maintaining the pressure drop across the panel at a desired level. More details regarding the structure and formation of the panels 210 are provided in U.S. Pat. Nos. 4,504,290 and 6,110,564, which are hereby incorporated by reference in their entirety. The panels 210 used in the illustrated embodiment can be obtained from Honeywell International Inc. as part number 203372, which are manufactured by Columbus Industries Inc. of Ashville, Ohio.

In the illustrated embodiment, the photocatalytic agent is a semiconductor metal oxide, more particularly titanium dioxide (in a mixture of the rutile and anatase forms), which is available from Degussa Chemical Company, Dusseldorf, Germany, under the product name P-25.

An example method for coating the panel including the expanded sheets with the photocatalyst is as follows. First, a 1 percent trisodium phosphate water solution or an Alconox solution is prepared in a container that has a circumference larger than the panel to be coated. The panel is then dipped in the solution and swirled to assure sufficient contact between the solution and the sheets contained in the panel (e.g., for five minutes or less). The panel is then removal from the solution and rinsed with deionized water to remove all trisodium phosphate, and the panel is allowed to air-dry or oven dried in a low temperature oven.

Second, the panel is wetted with a 10 percent sodium hydroxide (NAOH) solution for approximately 5 minutes. The sodium hydroxide is then rinsed off the panel with deionized water, and the panel is dried in a low temperature (e.g., 100 degree C.) oven for approximately 1 hour.

Third, the catalyst slurry is prepared. The amount of slurry that is needed is estimated based on the size of the container used for coating. Assuming that a 1 liter slurry is desired, 28.6 g ca. 95% sulfuric acid is added to 1000 g deionized water and mixed well. Then, 142.9 g P-25 catalyst is added to the solution, and the solution is mixed a sufficient amount of time to form a milky slurry. Preferably, the solution is allowed to mix for at least 2 hours, and more preferably 10, 12, or 24 hours.

Fourth, the panel is submerged in the catalyst slurry and swirled for a short amount of time, such as, for example, 5 seconds or less. The panel is then removed from the slurry, and the excess slurry is allowed to drain from the panel. The slurry is also drained from any hollow spaces in the frame of the panel. An air knife is used to remove excess slurry from the panel. The panel is then dried at 100 degrees C. for 30 minutes.

The sulfuric acid causes slightly dissolution or peptization of the titania and acts upon the expanded aluminum, thereby allowing the photocatalytic agent to adhere to the expanded sheets. It is, however, important to rapidly submerge and remove the panel from the slurry, as well as to use the correct concentration of sulfuric acid, as sulfuric acid can completely dissolve aluminum if submerged for an extended period of time or in high enough concentrations.

Fifth, the panel is placed in a Despatch oven and calcination is accomplished by heating the panel at between approximately 25 and 540 degrees C. over approximately 2.5 hours, then holding at approximately 540 degrees C. for approximately 1 hour, and then cooling back to room temperature over approximately 4 hours. After heating is completed and the panel is allowed to cool, the panel is removed from the oven.

Other semiconductive agents that absorb UV light can also be used, such as, for example, zinc oxide, cadmium sulfide, and zinc sulfide.

The filtering system 150 may exhibit one or more of the following advantages. The configuration of the wire support removes the angular dependence for the illuminated surface area, thereby enhancing the amount of surface area of the photocatalytic agent exposed to the UV light. Many prior designs included support structures forming channels through which the UV light could not pass and therefore activate the photocatalytic agent. Further, the geometry of the expanded sheets increases the fraction of the surface area that is illuminated by the UV lamp and also comes in contact with the air, thereby increasing filtration. In addition, the configuration of the panels reduces the pressure drop across them, allowing for enhanced airflow and the addition of supplemental filtering stages without significant loss in flow.

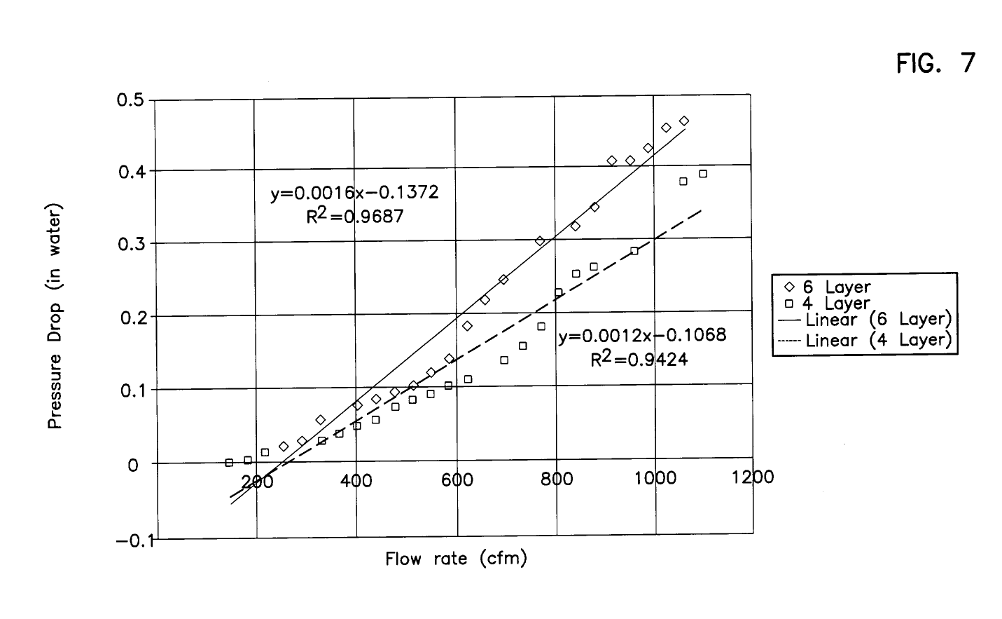

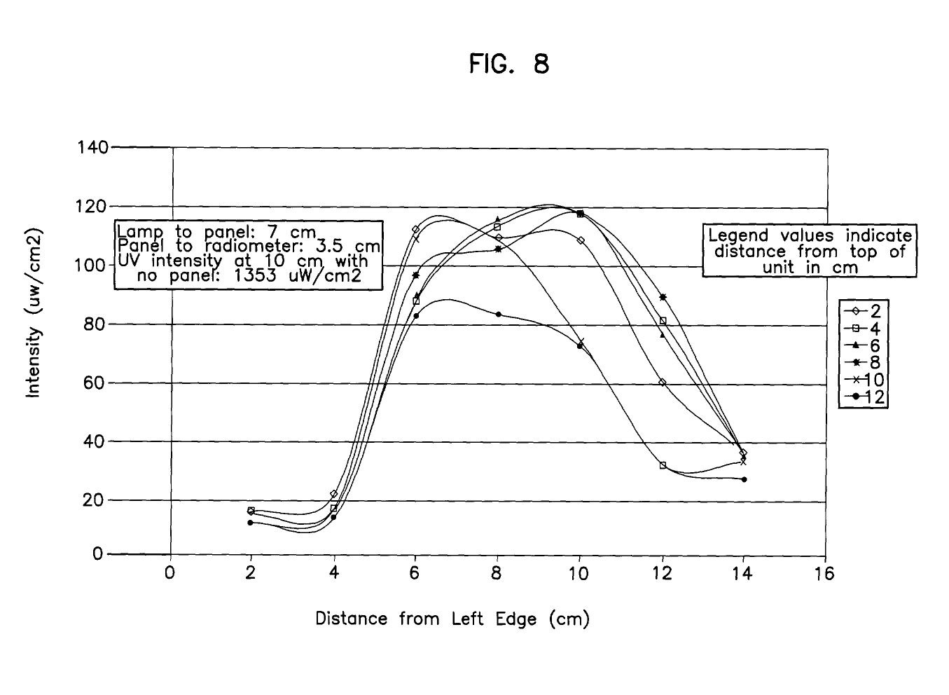

Referring now to FIGS. 7 and 8, two graphs are provided illustrating the pressure drop and ultraviolet transmittance, respectively, of example panels made in accordance with the present invention. The measurements illustrated in FIGS. 7 and 8 were performed on panels with four and six layers measuring 11.5 cm by 15 cm in size. In FIG. 7, the measured pressure drop is plotted for two panels, a first panel including four expanded sheets and a second panel including six expanded sheets. Both panels exhibited pressure drops well below maximum tolerated levels. For example, in one application, the maximum allowable pressure drop is approximately one inch water column, and typical flow rates are approximately 1000-4000 cfm. Linear equations approximately describing the experimental results are stated on the graph for each type of panel.

In FIG. 8, the measured ultraviolet transmittance through a panel having six layers of expanded sheets is shown, the panel measuring 11.5 cm by 15 cm. Each panel is mounted 7 cm in front of two ultraviolet lamps and the light intensity is measured using a radiometer spaced 3.5 cm from the panel. Without a panel, the light intensity was 1353 μW/cm². The legend values indicate the distance from the top of the panel in centimeters where the intensity measurements were made. As illustrated, approximately 10 percent of the ultraviolet light was transmitted though the six-layered panel near the center of the panel, confirming that the panel allows light to be transmitted through the various layered expanded sheets, so that the light reaches and activates a substantial portion of the panel.

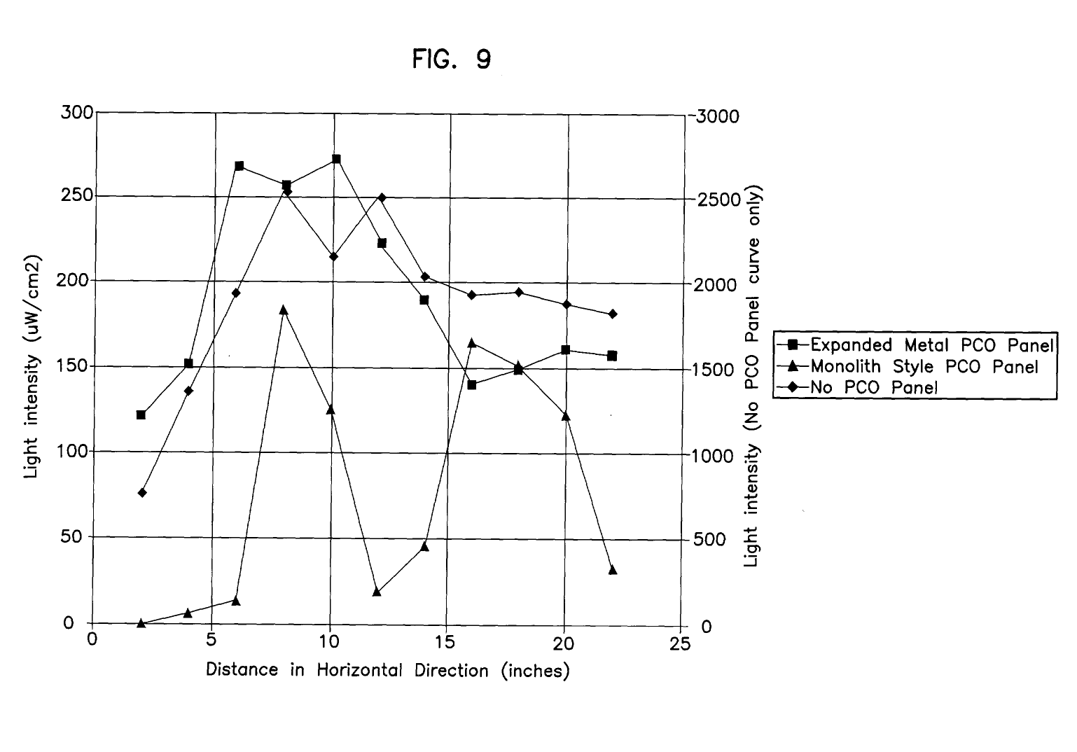

Referring now to FIG. 9, light transmission for several different filtering configurations are compared. Specifically, light intensity measurements are taken for three configurations: (1) no panel in place; (2) a monolith photocatalytic panel design; (3) a panel including four expanded sheets made in accordance with the present invention. In the monolith panel design, the support is a sheet of corrugated cardboard approximately 2 cm thick, oriented so that the light shines down the corrugations. The monolith panel is impregnated with titanium dioxide and has more surface area than the panel including the expanded sheets. As shown in FIG. 9, there is dramatically less light transmitted through the monolith panel design as compared with no panel and the panel including the expanded sheets. As previously noted, the greater the amount of light that travels through a panel, the greater the surface area of photocatalytic agent that is activated, and, therefore, the greater the filtering efficiency.

Table 1 below provides a comparison of performance for removing an airborne contaminant from an air stream between a panel including four expanded sheets made in accordance with the present invention and the monolith panel design. Although higher intensity ultraviolet lights are used with the monolith design, significantly less of the airborne contaminant (acetaldehyde in the example shown in Table 1) is converted. The sixth column titled “Conc. (Off)” shows the concentration of acetaldehyde passing through the unit when it is turned off.

TABLE 1
Conversion of acetaldehyde passing through a
photocatalytic device comprised of UV lamps
between two parallel panels.

| | | | | | | |
|---|---|---|---|---|---|---|
| | | | | Air Flow | Conc. | |
| | Number | Lamp | Number | Rate | (Off) | % |
| Run | of Lamps | Wattage | Panels | (cfm) | (ppm.) | Removal |

Expanded Metal Panels

| | | | | | | |
|---|---|---|---|---|---|---|
| 1 | 2 | 36 | 2 | 1359 | 0.21 | 2.62% |
| 2 | 2 | 36 | 2 | 1366 | 0.19 | 12.14% |
| 3 | 2 | 36 | 2 | 1360 | 0.52 | 4.08% |
| 4 | 2 | 36 | 2 | 1361 | 0.19 | 13.99% |
| 5 | 2 | 36 | 2 | 1836 | 0.17 | 20.18% |
| 6 | 2 | 36 | 2 | 1839 | 0.32 | 12.99% |
| 7 | 2 | 36 | 2 | 1365 | 0.20 | 14.08% |
| 8 | 2 | 36 | 2 | 1502 | 0.26 | 19.08% |
| 9 | 2 | 36 | 2 | 995 | 0.32 | 18.65% |
| 10 | 2 | 36 | 2 | 996 | 0.25 | 20.18% |
| 11 | 2 | 36 | 2 | 1499 | 0.19 | 16.00% |

Cardboard Monolith
Panels

| | | | | | | |
|---|---|---|---|---|---|---|
| 13 | 2 | 55 | 2 | 1062 | 0.36 | −0.49% |
| 14 | 4 | 55 | 2 | 1062 | 0.35 | 2.71% |

Although a negative percentage for removal of acetaldehyde is shown for trial run 13 with the cardboard monolith panel, this negative percentage is within the error range of zero (0) percent for the measured values.

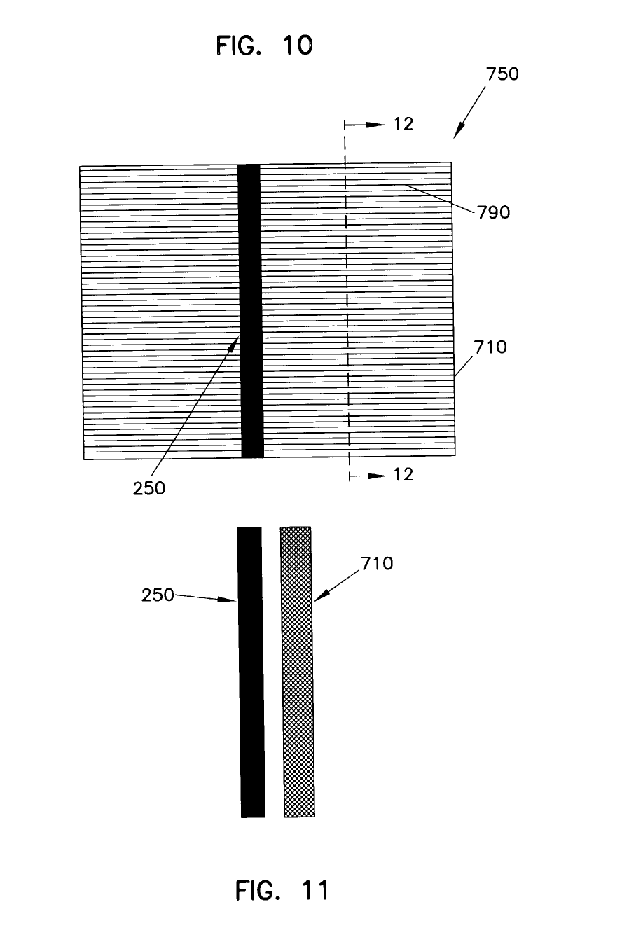

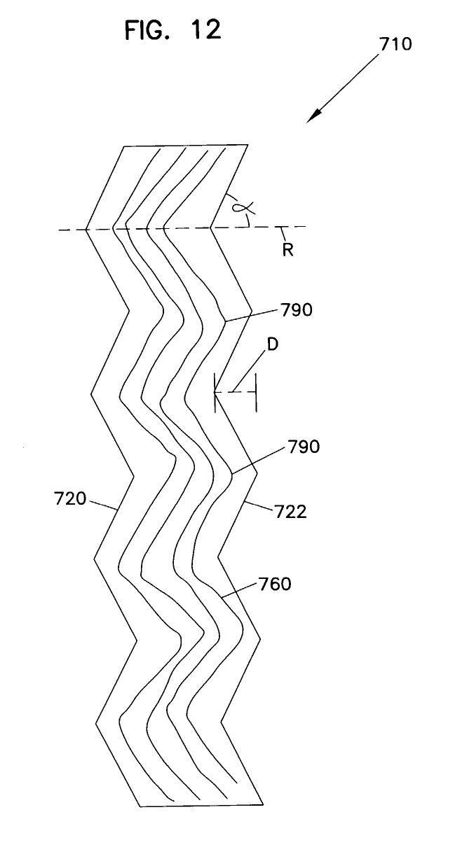

Referring now to FIGS. 10-12, a second embodiment of a filtering system 750 is shown. The system includes a panel 710 and the lamp 250. The panel 710 is similar to the panels 210 described above, except that the expanded sheets 760 positioned in the panel 710 include pleats 790 running approximately parallel to one another. In the embodiment shown, an optimum pleat depth D of 0.25-2 inches is used. In other embodiments, a pleat depth D of 1 inch is used, with the number of pleats on each panel spanning between 10 and 18. An angle a formed between the folded surface of a pleat 790 and a line R running perpendicular to the pleated panel may be varied to optimize the surface area of the expanded sheets that is exposed to the fluid. In one example, the angle ca is less than 10 degrees, although the angle can be increased (e.g., 20 degrees, 30 degrees, etc.), with a resulting decrease in total surface area for the sheet. In the embodiment shown, the front and rear support surfaces 720 and 722 of the panel 710 also include a pleated structure.

The lamp 250 is positioned so that the lamp extends perpendicular to the direction of the pleats 790. In the illustrated embodiment, the lamp 250 extends at least the majority of the length of the panel 710.

The filtering system 750 may be advantageous because the pleats allow for a greater cross-sectional area of the panel to be exposed to the UV light and air flowing through the panel, thereby increasing filtering capacity.

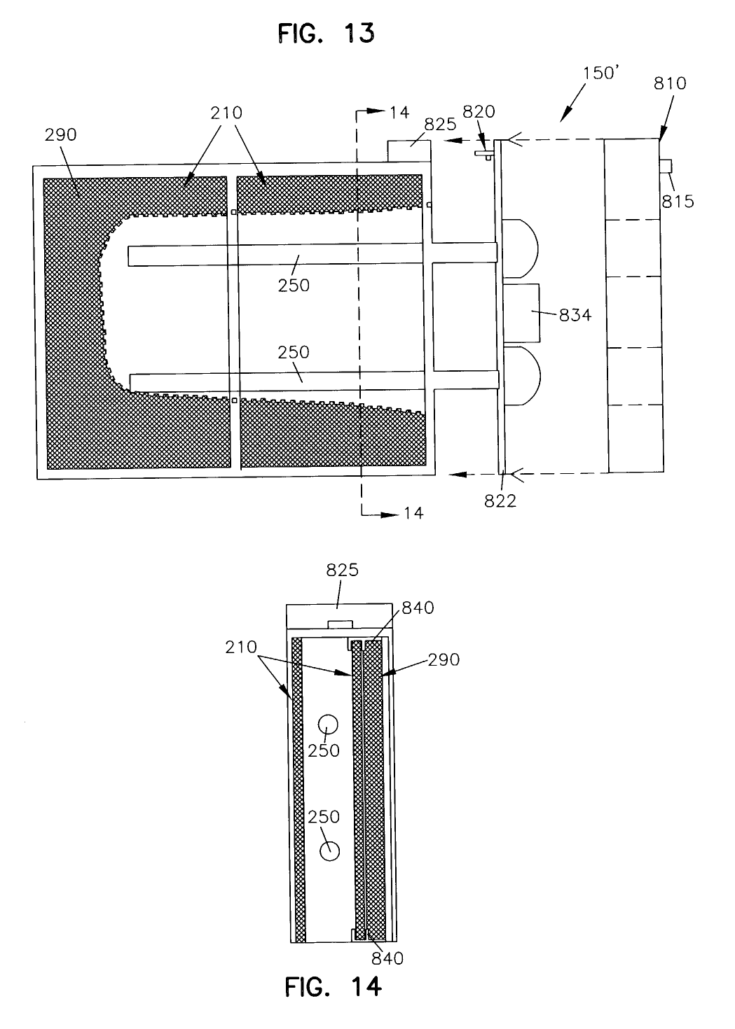

Referring now to FIGS. 13 and 14, another embodiment of a filtering system 150′ is shown. The filtering system 150′ is similar to system 150, except for the addition of a removable door shell 810 and lamp holder 822 that allow for ease in accessing the panels 210 and the lamps 250, and the horizontal orientation of the lamps 250. The shell 810 includes a handle 815 to allow a user to grasp and remove the shell 810. The lamp holder 822 includes a ballast 834. The lamp holder 822 also includes a latch 820 that is received in a latch box 825 to retain the lamp holder 822 in place. Brackets 840 (see FIG. 14) are used to retain the filter 290 and panel 210 in place within the filtering system 150′.

Alternative embodiments of the filtering systems are also possible. For example, instead of two ultraviolet lamps, one lamp may be used, or greater than two lamps, such as three or four lamps, can be used. The lamps may also be placed on both sides of a panel, thereby illuminating a greater surface area and thereby increasing filtering efficiency. A panel may be placed on both sides of a lamp or pair of lamps. If a lamp or lamps are placed on both sides of a panel, then a thicker panel may be used, such as panel with 8-10 expanded sheets, since the illumination will be coming from both directions. In another embodiment, the filtering system may be used in a stand-alone unit, rather than a HVAC system, and one or more fans can be used to move air through the filtering system. In addition, although the illustrated embodiments describe a particular number of panels and lamps used in the filtering system, one skilled in the art can vary the number of panels and lamps used in a particular filtering system as desired. For example, multiple sets of panels and lights can be used in a filtering system, where the panels and lamps are alternated in the direction of the airflow, thereby creating multiple stages of filtration. For example, two, three or four sets of a panel and lamps may be included in the filtering system. In another example, five sets of a panel and a lamp or pair of lamps are placed next to each other with a sixth panel placed at the end of the group next to the exposed lamp.

Although the illustrated embodiments are described with respect to filtering of air, other applications are also possible. For example, the filtering systems disclosed herein may be used in filtration of various gas or vapor phase applications. These applications include mixtures of nitrogen, oxygen, and water, containing any of carbon dioxide, carbon monoxide, ammonia, helium, neon, argon, krypton, fluorocarbons, sulfur hexafluoride, nitrogen trifluoride, silicon tetrafluoride, or mixtures of these components, and contaminated with undesirable impurities including volatile organic compounds, nitrogen oxides, or hydrogen sulfide.

The present invention should not be considered limited to the particular examples or materials described above, but rather should be understood to cover all aspect of the invention as fairly set out in the attached claims. Various modifications, equivalent processes, as well as numerous structures to which the present invention may be applicable will be readily apparent to those of skill in the art to which the present invention is directed upon review of the instant specification.

What is claimed is:

1. A panel for a fluid cleansing system, the panel comprising:
a support structure positioned generally perpendicular to a fluid stream and defining an interior space, the support structure defining a plurality of apertures to allow fluid to flow through the apertures;
at least one expanded sheet positioned within the space, the expanded sheet defining a plurality of expanded slits; and
a photocatalytic agent coupled to the expanded sheet to oxidize airborne contaminants when exposed to a light source.

2. The panel of claim 1, wherein the expanded sheet is made of metal.

3. The panel of claim 1, wherein the expanded sheet includes aluminum.

4. The panel of claim 1, further comprising a plurality of expanded sheets positioned within the space, the plurality of expanded sheets each defining a plurality of expanded slits.

5. The panel of claim 1, wherein each of the plurality of expanded slits is formed by stretching the expanded sheet in at least one direction.

6. The panel of claim 1, wherein each of the plurality of expanded slits defines a generally diamond-shaped opening.

7. The panel of claim 1, wherein the panel is shaped to include a plurality of pleats, each of the plurality of pleats being generally parallel to one another and generally perpendicular to a longitudinal direction of the light source.

8. The panel of claim 1, wherein the support structure includes two wire sheets positioned generally parallel to one another to define the space.

9. The panel of claim 1, wherein the photocatalytic agent comprises a semiconductor metal oxide.

10. The panel of claim 9, wherein the semiconductor metal oxide includes titanium dioxide.

11. The panel of claim 1, wherein each of the plurality of expanded slits is generally parallel to one another.

12. A system for cleansing a fluid stream, comprising:
at least two panels positioned generally parallel to one another, perpendicular to the fluid stream, and defining a first space therebetween, each panel including:
a support structure including two support sheets positioned generally parallel to one another, perpendicular to a fluid stream, and defining a second space therebetween, each of the two support sheets defining a plurality of apertures to allow fluid to flow through the apertures;
at least one expanded sheet positioned within the second space, the expanded sheet defining a plurality of expanded generally parallel slits; and
a photocatalytic agent coupled to the expanded sheet to oxidize airborne contaminants when exposed to a light source; and
an ultraviolet light source positioned in the first space between the two panels.

13. The system of claim 12, wherein the ultraviolet light source is a first ultraviolet light source, and the system further comprises a second ultraviolet light source positioned generally parallel to the first ultraviolet light source in the first space.

14. The system of claim 12, wherein each of the expanded sheets is made of metal.

15. The system of claim 12, wherein each of the expanded sheets includes aluminum.

16. The system of claim 12, wherein each of the two panels further includes a plurality of expanded sheets positioned within the second space, the plurality of expanded sheets each defining a plurality of expanded generally parallel slits.

17. The system of claim 12, wherein each of the plurality of expanded generally parallel slits of each expanded sheet is formed by stretching the respective expanded sheet in at least one direction.

18. The system of claim 12, wherein each of the plurality of expanded generally parallel slits of each expanded sheet defines a generally diamond-shaped opening.

19. The system of claim 12, wherein each of the at least two panels is shaped to include a plurality of pleats, each of the plurality of pleats being generally parallel to one another and perpendicular to a longitudinal direction of the ultraviolet light source.

20. A method for cleansing a fluid stream, comprising:
providing a sheet;
cutting a plurality of slits in the sheet;
stretching the sheet to expand each of the plurality of generally parallel slits;
providing a support around the sheet to form a first panel;
coating the sheet with a photocatalytic agent;
disposing the first panel perpendicular to the fluid stream; and
exposing the first panel to ultraviolet light to activate the photocatalytic agent.

21. The method of claim 20, further comprising selecting a metal sheet as the sheet.

22. The method of claim 20, further comprising:
providing a second panel including a sheet with a plurality of expanded slits and a support structure;
positioning the second panel generally parallel to the first panel to define a space therebetween; and
providing an ultraviolet light source positioned between the first and second panels.

23. The method of claim 20, further comprising shaping the first panel to include a plurality of pleats, each of the plurality of pleats being generally parallel to one another and perpendicular to a longitudinal direction of the ultraviolet light source.

24. The method of claim 20, further comprising providing a plurality of expanded sheets positioned adjacent to one another and the sheet, each of the plurality of sheets defining a plurality of expanded slits.

25. The method of claim 20, wherein the cutting step comprises cutting the plurality of slits so that each slit is generally parallel to one another.

26. The method of claim 20, wherein the coating step comprises:
preparing a slurry including the photocatalytic agent;
submerging the first panel, including the sheet, in the slurry;
removing the first panel from the slurry.

27. The method of claim 26, wherein the slurry includes sulfuric acid.

28. The method of claim 26, wherein the preparing step including mixing the slurry for at least two hours.

29. The method of claim 20, further comprising slightly dissolving the sheet to increase adherence of the photocatalytic agent to the sheet.
